(19) United States
(12) Patent Application Publication
(10) Pub. No.: US 2008/0140035 A1
(43) Pub. Date: Jun. 12, 2008

(54) Male Urinary Incontinence Device and Method

(76) Inventors: Judith Gadzik, Westport, CT (US); Susan MacArthur, Farmington, CT (US)

Correspondence Address: LAW OFFICE OF MICHAEL A. BLAKE, 112 BROAD STREET, MILFORD, CT, 06460, US

(21) Appl. No.: 11/608,031

(22) Filed: Dec. 7, 2006

(51) Int. Cl. A61F 5/44; A61F 13/15

(52) U.S. Cl. 604/349; 604/385.01

(57) ABSTRACT

A method for using male urinary incontinence device, a method for packaging a male urinary incontinence device, and a male urinary incontinence device comprising: a generally square shaped pad, where the generally square shaped pad comprises: a first corner located on the generally square shaped pad; a second corner located on the generally square shaped pad; a third corner located on the generally square shaped pad; a fourth corner located on the generally square shaped pad; a first layer that is water absorbent; a second layer attached to the first layer, and wherein the second layer is water impermeable; a first guide line visibly located on the first layer, and extending from generally the first corner to generally the third corner; a second guide line visibly located on the first layer, and extending from generally the second corner to generally the fourth corner; a guide figure of appropriate configuration visibly located on the first layer, superimposed on the first line, situated between the first corner and the center; an elastic band attached to the first layer at generally the third corner.

### TECHNICAL FIELD

This invention relates to a sanitary disposable pad and in particular to such a pad for use by males.

### BACKGROUND

The male urinary tract is analogous to ordinary household plumbing. For instance, as a hollow tank stores water in a home, a muscular sac, called the bladder, is a reservoir for urine. Additionally, as a faucet attached to the side of the storage-tank regulates water flow from the tank, a muscle sphincter leading from the bladder regulates urine flow. When the sphincter tightens (analogous to a faucet being shut off), there is no flow. When the sphincter relaxes (analogous to a faucet being opened), there is free flow of liquid. Just as a hose, attached to the faucet, conducts water away from the storage tank, a collapsible tube called the urethra conducts urine from the bladder to the tip of the penis.

If a hose is attached to a faucet on one end, but has no nozzle at the other end, water remaining within the hose can trickle out, even after the faucet has been shut off. Unfortunately, the urethra is a hose without a nozzle. Even when the muscle sphincter tightens, stopping further emptying of the bladder, urine already within the urethra will continue to leak out at the tip of the penis. This may lead to the soiling the undergarments and/or an unpleasant feeling of wetness. This leakage is known as “natural urinary incontinence”. Natural urinary incontinence can cause unpleasant odor, wetness and staining of undergarments, as well as over garments such as pants, and shirts.

It would therefore be desirable to have a device that can prevent the above mentioned effects of natural urinary incontinence.

### SUMMARY

The disclosed invention relates to a male incontinence device comprising: a generally square shaped pad, where the generally square shaped pad comprises: a first corner located on the generally square shaped pad; a second corner located on the generally square shaped pad; a third corner located on the generally square shaped pad; a fourth corner located on the generally square shaped pad; a first layer that is absorbent; a second layer attached to the first layer, and wherein the second layer is water impermeable; a first guide line visibly located on the first layer, and extending from generally the first corner to generally the third corner; a second guide line visibly located on the first layer, and extending from generally the second corner to generally the fourth corner; a guide figure, situated between the first corner and the center, visibly located on the first layer, superimposed over the first guide line, and shaped like, but not limited to, a bullet, phallus, cylinder or rectangle; an elastic band attached to the first layer at generally the third corner.

The disclosed invention also relates to a method of applying a male urinary incontinence device to a user's phallus. The method comprises orienting the male incontinence device such that a first layer is facing up, a second layer is facing down, and a solid line generally between corners one and three is oriented perpendicular to an imaginary line connecting the user's hips, and an elastic band and third corner are oriented at generally the 12 o'clock position relative to the user; moving the elastic band superiorly and off the first layer; placing the phallus on the first layer generally along the first line, over the first corner and guide figure, and below the second line; folding the male urinary incontinence device generally along the second line and over the phallus, transposing the third corner; folding a fourth corner over the phallus and adjacent to a third corner; folding a second corner over a segment containing the fourth corner, such that the second corner is adjacent to the third corner; looping the elastic band from front to back over the phallus and the male incontinence device such that the that elastic band is below glans; twisting the elastic band 180 degrees, creating a second loop; and placing the second loop over and around the male urinary incontinence device and phallus such that the second loop is below the glans.

In addition, the disclosed invention relates to a method of packaging a male urinary incontinence device, comprising: orienting the male incontinence device such that a first corner is pointed away from a packager; folding the first corner over until it covers a third corner; folding a fourth corner over until it meets the first corner and creates a fifth corner; folding a fifth corner so that it generally meets a midpoint between a second corner and the first corner; and folding a second corner toward the fourth corner.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is top view of a disclosed male urinary incontinence device;

FIG. 2 is a top view of another embodiment of a disclosed male urinary incontinence device;

FIG. 3 shows a top view of an open pocket of a water absorbing material;

FIG. 4 shows a perspective view of a semi-opened pocket of a water absorbing material;

FIG. 5 shows a top view of a closed pocket of a water absorbing material;

FIGS. 6 through 14 shows the steps of applying the male urinary incontinence device to a phallus;

FIGS. 15 through 21 shows the steps of folding the male urinary incontinence device for packaging;

FIG. 22 is a flowchart showing a method of applying the male urinary incontinence device to a phallus; and

FIG. 23 is a flowchart showing a method of folding the male urinary incontinence device for packaging.

### DETAILED DESCRIPTION

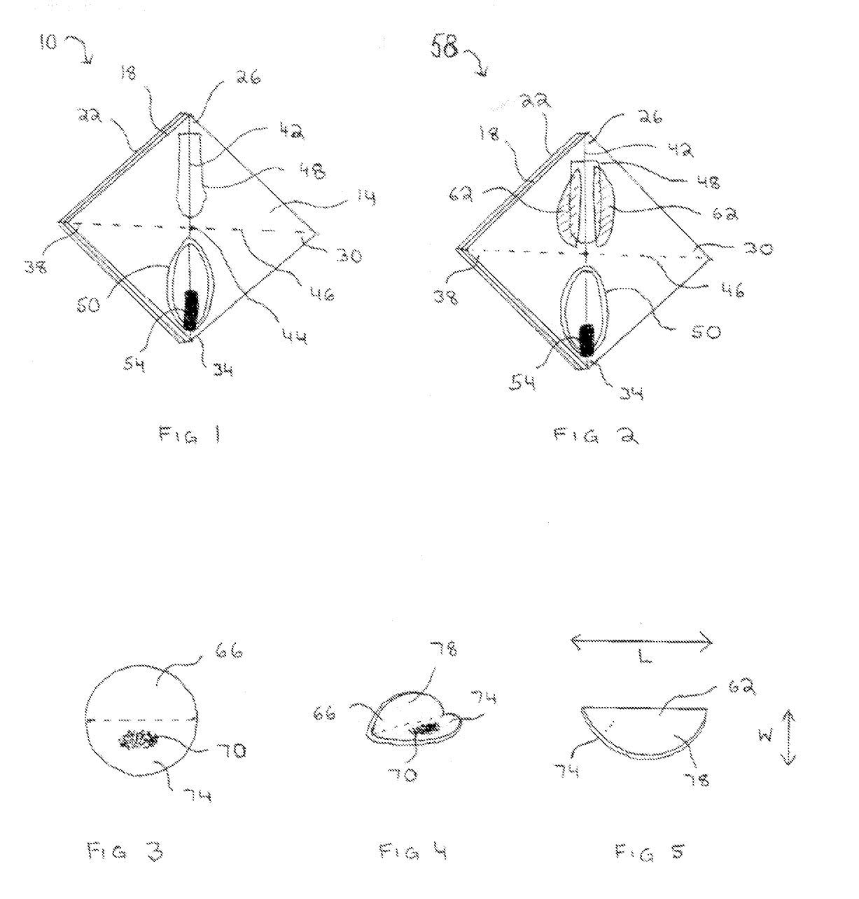

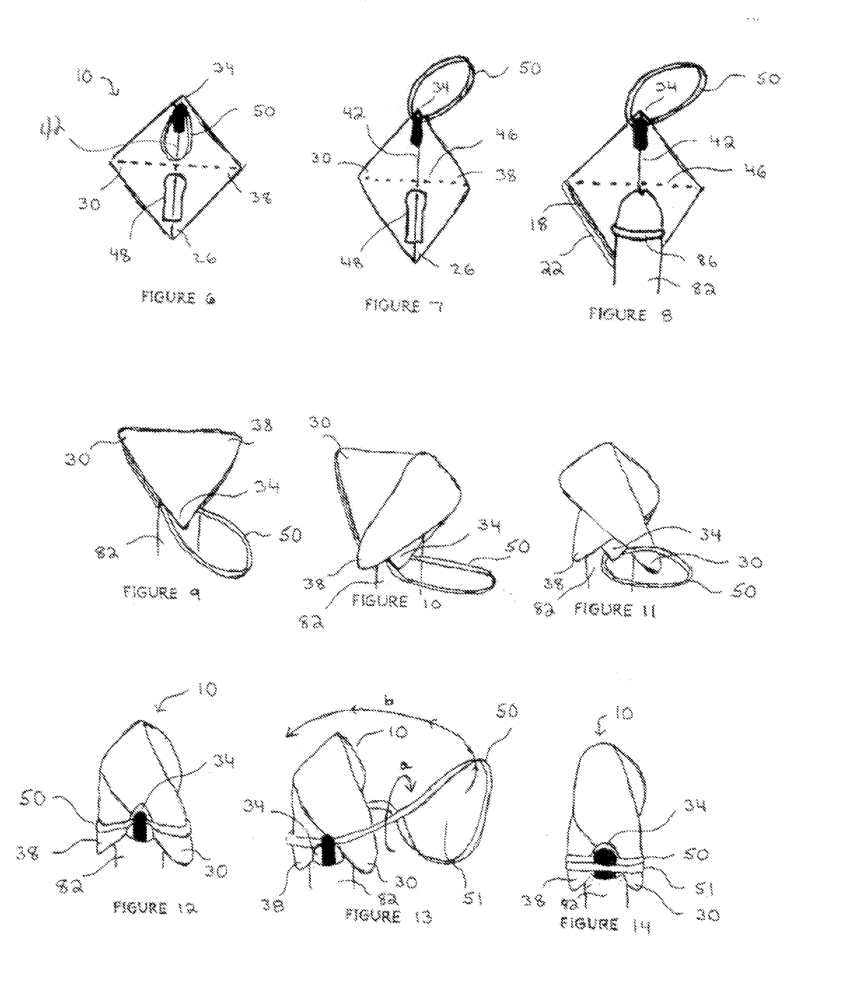

FIG. 1 shows one embodiment of the disclosed male urinary incontinence device 10 The device comprises a generally square shaped pad 14. The pad 14 comprises a first layer 18 and a second layer 22. The second layer 22 is a water-impermeable material, such as but not limited to plastic, rubber, or nylon. The first layer 18 is an absorbent material, such as but not limited to tissue paper, paper towel, paper napkin, cotton cloth, and nylon cloth. The first layer 18 may be two-ply, single-ply, or triple-ply. Each side of the pad may be about 3 inches (7.6 cm) to about 10 inches (25.4 cm), and preferably about 5.9 inches (15 cm). The first layer 18 may be attached to the second layer 22 by using an adhesive between the two layers, or by using other attachment means such as but not limited to plastic staples, sealing, heat sealing, and sewing the two layers 18, 22 together.

The pad 14 has four corners, a first corner 26, a second corner 30, a third corner 34, and a fourth corner 38. The first layer 18 has a first line 42 starting at generally the first corner 26 and ending at generally the third corner 34. The first layer 18 has a second line 46 starting at generally the second corner 30 and ending at the fourth corner 38. The first line 42 may be visibly distinguished from the second line 46. The means of visibly distinguishing the lines 42, 46 may be that one of the two lines 42, 46 may be a solid line, and the other line may be a dashed, or dotted line, or in other embodiments, one of the two lines 42, 46 may be a first color, and the other line may be a second color. In still other embodiments, lines 42, 46 may be indistinguishable from each other. The lines 42, 46 are guide lines to assist the user in the proper folding and applying the male urinary incontinence device 10. The first layer 18 may have a guide FIG. 48, superimposed on first line 42, between the first corner 26 and the center 44, with the shape of, but not limited to, a bullet, phallus, rectangle or cylinder. The guide FIG. 48 assists in the application of the male urinary incontinence device. In other embodiments, the guide FIG. 48 may be omitted. Attached to the first layer 18 at or near the third corner 34 is an elastic band 50. The elastic band 50 may have a diameter of about 2 inches. The elastic band may be made out of any suitably elastic material, including but not limited to a natural rubber, latex rubber, plastic, nylon, spandex. The elastic band 50 is attached to the first layer 18 via at least one attachment means 54. The at least one attachment means 54 may include any suitable attaching means, such as, but not limited to: metal staples, plastic staples, adhesive, adhesive tape, and stitching. In the disclosed embodiment shown in FIG. 1 the attachment means 54 is shown as an adhesive strip, attaching the elastic band 50 to the first layer 18.

FIG. 2 shows another embodiment of the male urinary incontinence device 58. In this embodiment, there is at least one pocket 62, containing a water absorbing material 70 (not shown in this Figure), attached to the first layer 18 near the first corner 26. As drawn in the figure, the pocket 62 has an oblong shape, with a length “L” greater than its width “W” (see FIG. 5), but can also be shaped like a triangle, rectangle, circle square, or any appropriate configuration. The length L is generally parallel to the first line 42. The water absorbing material 70 (see FIGS. 3 and 4) may be any suitable material for the absorption of urine, such as but not limited to Polyacrylamide powder. FIGS. 3 through 5 shows how the pockets 62 may be made. FIGS. 3 shows a circular disk of water permeable material 66, such as but not limited to cotton. Water absorbing material 70 is placed on a first half 74 of the disk of material 66. The second half 78 of the disk of material 66 generally has no water absorbing material 70 placed upon it. FIG. 4 shows the second half 78 of the disk of material 66 being folded over the first half 74. FIG. 5 shows the two halves 74, 78 of material sealed with each other, holding the water absorbing material 70 (not visible in this view) between the two halves, and forming a pocket 62. The two halves 74, 78 may be sealed with any sealing means, including but not limited to adhesive sealing, and stitched sealing. This embodiment, shown in FIG. 2, may be used for individuals who have excessive amounts of leakage. Some advantages of using Polyacrylamide powder in this way are that the powder absorbs almost 500 times its weight in water and is safe for human external use.

FIGS. 6 through 14 show how the male urinary incontinence device (either of the embodiments disclosed so far) may be applied to a phallus. The user should open the male incontinence device with the first layer 18 facing up, the second layer 22 facing down, the solid line 42 oriented perpendicular to an imaginary line connecting the user's hips, and the elastic band 50 and third corner 34 oriented at generally the 12 o'clock position on an imaginary clock, as shown in FIG. 6. (Notice that in the orientation now described, the embodiments in FIGS. 1 and 2 have been rotated 180 degrees, so that the third corner 34 is farthest away from the user and the first corner 26 is closest.) The elastic band should then be moved superiorly, off the first layer 18, as shown in FIG. 7. Next, the phallus 82 should be placed on the first layer 18, over the first corner 26 and guide FIG. 48, generally along the first line 42, but generally below the second line 46, as shown in FIG. 8. Next, the male urinary incontinence device 10 should be folded, generally along the second line 46, over the phallus 82, as shown in FIG. 9. Next, the fourth corner 38 should be folded over towards the third corner 34 and the phallus 82, then wrapped slightly around the penile shaft, as shown in FIG. 10. Next, the second corner 30 is folded over the fourth 38, over the first corner 34 and over the phallus 82, then slightly wrapped around the penile shaft, as shown in FIG. 11. Next, the elastic band 50 is looped over the phallus 82 and the folded male urinary incontinence device 10, so that the band 50 rests below the covered glans 86 of the phallus 82 (FIG. 8 shows the location of the glans 86 on the phallus 82), as shown in FIG. 12. In FIG. 13, the elastic band 50 is twisted generally about 180 degrees, forming a second loop 51. Next, the second loop 51 is placed over and around the folded male urinary incontinence device 10 and the phallus 82 below the covered glans 86, see FIG. 14. If desired, for further tightening, a third loop, or as many extra loops as needed, may be fashioned

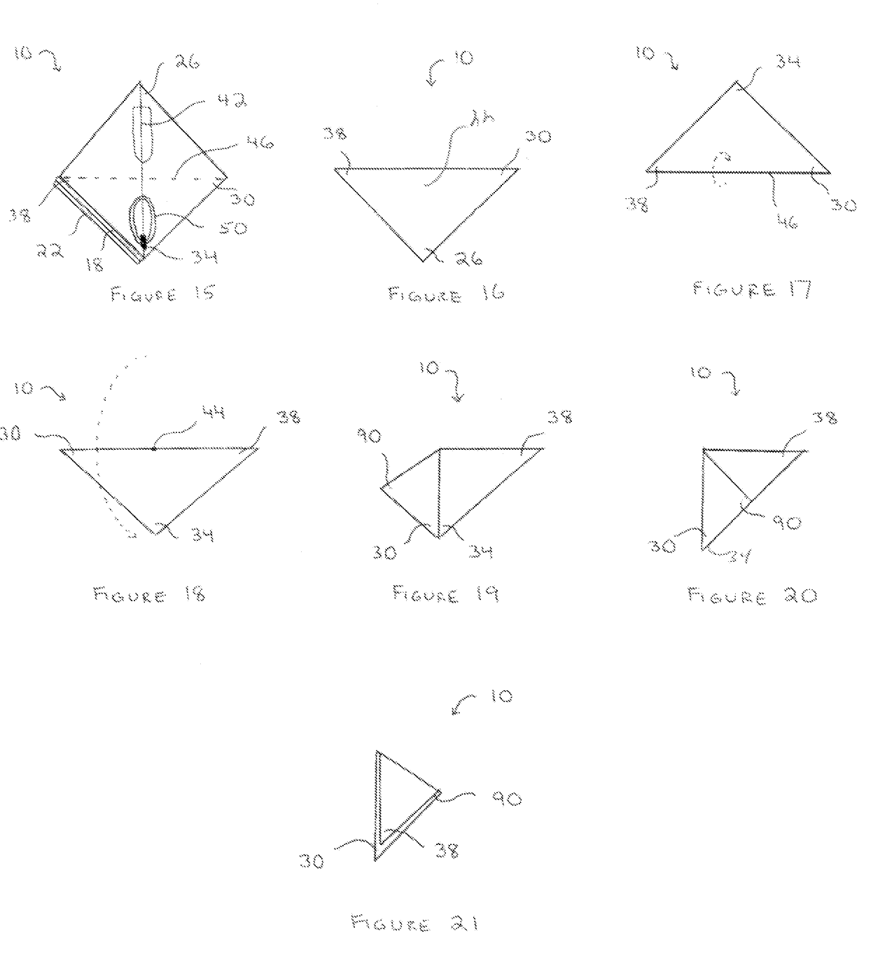

Another advantage of the disclosed male urinary incontinence device is that it may be packaged so that it is small and compact and may be carried in a wallet, pocket, glove compartment, etc. FIGS. 15 through 21 show how the disclosed male urinary incontinence device may be folded and thereafter packaged into a compact configuration. First you start with a male urinary incontinence device 10 arranged as shown in FIG. 15, with the first corner 26 pointed away from the user, folding machine, or other folding device. Next, the device 10 is folded along line 46, until the first corner 26 covers the third corner 34, as shown in FIG. 16, thus placing the elastic band 50 and the water absorbent layer 18 within the impermeable layer 22. The entire unit 10 is flipped over, along the axis of the second line 46 between the second 30 and fourth 38 corners, so that the third corner 34 if pointing away from the user, as in FIG. 17. The entire device 10 is rotated 180 degrees around the center 44, as in FIG. 18, so that the third corner 34 now points toward the user. Next, the second corner 30 is folded over until it meets the third corner 34, as shown in FIG. 19. At this point, a new fifth corner 90 is formed, while the second corner 30 covers the left half of third corner 34. Next, the fifth corner 90 is folded so that it generally meets the midpoint of the male urinary incontinence device 10 between the fourth corner 38 and the third corner 34, as shown in FIG. 20, so that the second corner 30 now completely covers third corner 34. Next, as shown in FIG. 21, the fourth corner 38 is folded toward the third corner 34, giving the male urinary incontinence device 10 a final compact triangular shape, which then may be further packed with shrink wrap, cellophane, or boxed.

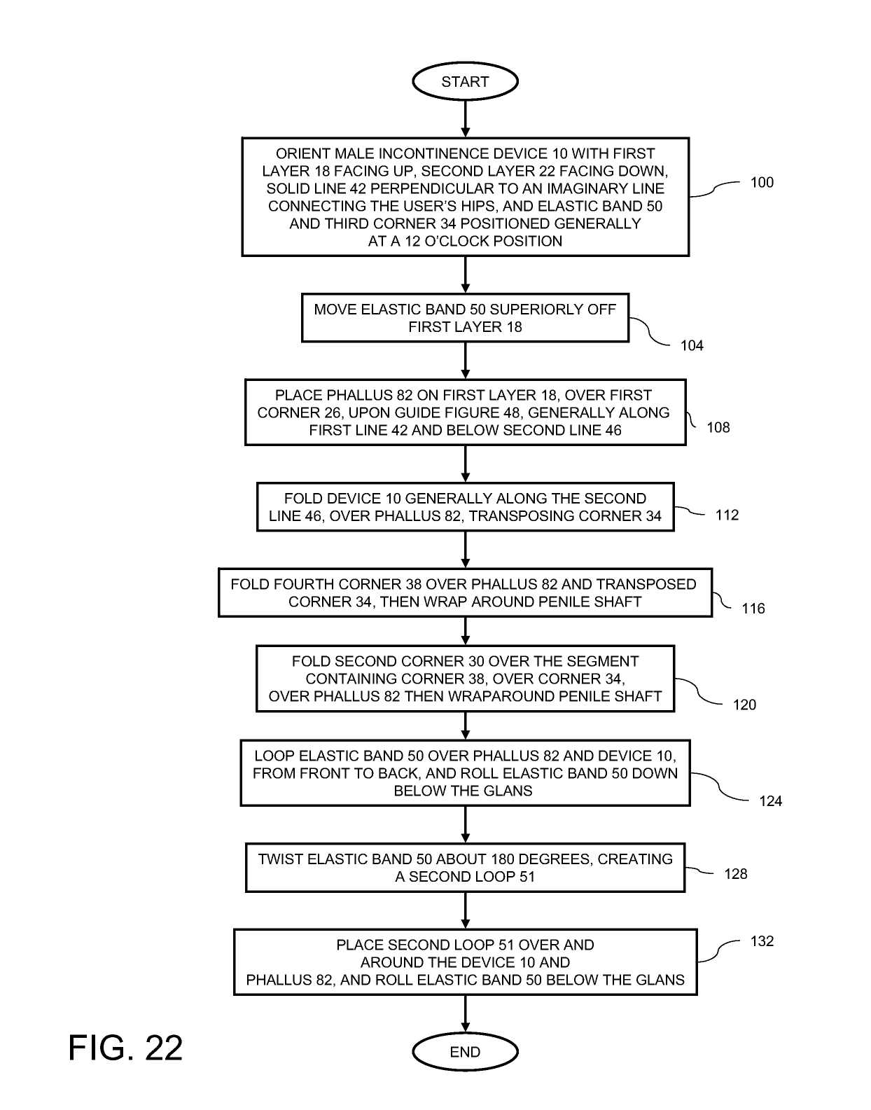

FIG. 22 shows a method of putting on the male urinary incontinence device 10 At act 100, the user orients the male urinary incontinence device with the first layer 18 facing up, the second layer facing down 22, the solid line 42 oriented perpendicular to an imaginary line connecting the user's hips, and the elastic band 50 and the third corner 34 oriented generally at a point farthest from the user. At act 104, the user moves the elastic band 50 superiorly, off the first layer 18. At act 108, the user places his phallus 82 on the first layer 18, generally along the first line 42, over the first corner 26 and the guide figure, and below the second line 46. At act 112, the user folds the male urinary incontinence device 10 generally along the second line 46, over his phallus 82. At act 116, the user folds the fourth corner 38 over the third corner 34, over his phallus 82, then slightly wraps the third corner 38 around the penile shaft. At act 120, the user folds the second corner 30 over the segment containing the fourth corner 38, over the third corner 34 and over the phallus 82, then slightly wraps corner 30 around the penile shaft. At act 124, the user loops the elastic band 50 over his phallus 82 and the folded male urinary incontinence device 10, from front to back, so that elastic band 50 is below the covered glans 86. At act 128, the user twists the elastic band 50 fully 180 degrees, creating a second loop 51. At act 132, the user places the second loop 51 over and around the folded male urinary incontinence device 10 and his phallus 82 below the covered glans 86.

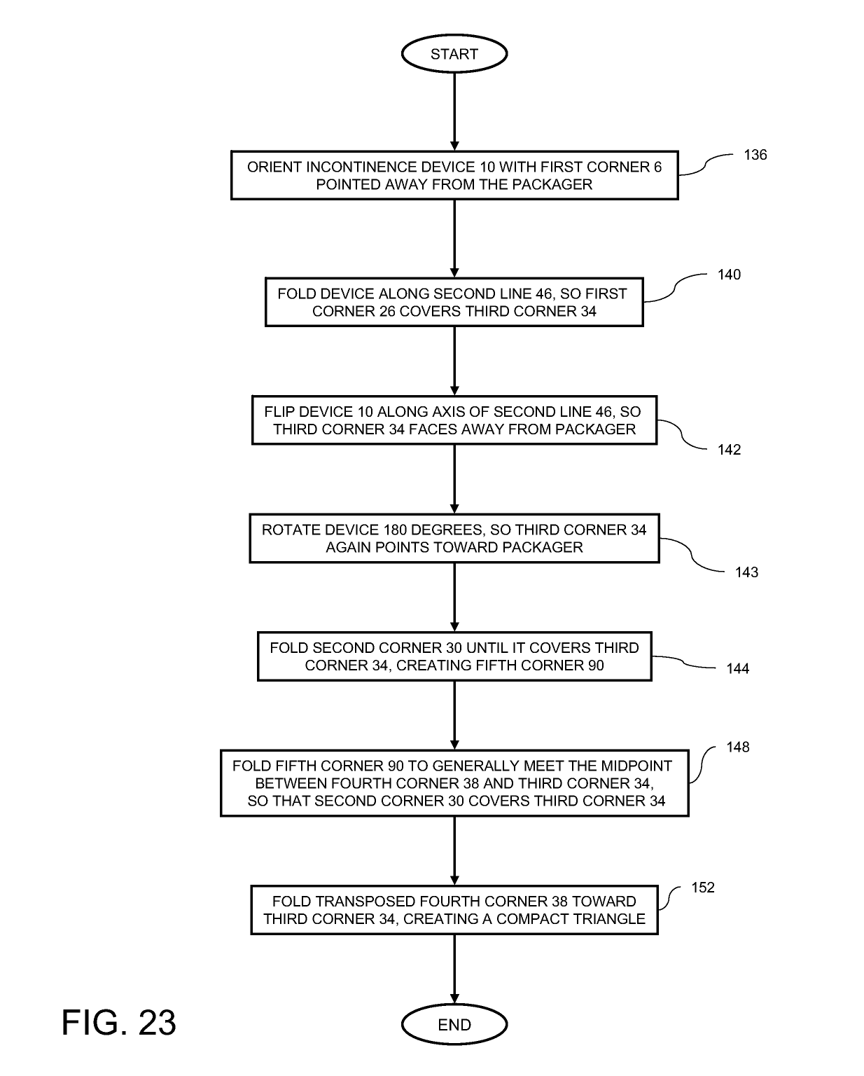

FIG. 23 shows a method of configuring a male urinary incontinence device 10 for packaging. At act 136, a packager orients the male incontinence device 10 such that the first corner 26 points away from the packager. At act 140, the packager folds the first corner 26 over until it covers the third corner 34. At act 142, the packager flips the device over, along the axis of the second line 46, so that the third corner points away from the packager. At act 143, the packager rotates the device 10 fully 180 degrees around the center 44, so that the third corner again points toward the packager. At act 144, the packager folds the second corner 30 over until it covers the left half of the third corner 34, creating a fifth corner 90. At act 148, the user folds the fifth corner 90 so that it generally meets at a midpoint on the male urinary incontinence device 10 between the fourth corner 38 and the right half of third corner 34, so that the second corner 30 now completely covers the third corner 34 after folding. At act 152, the packager folds the fourth corner 38 toward the third corner 34, producing a compact, triangular unit 10.

The disclosed male urinary incontinence device 10 has many advantages. The disclosed male urinary incontinence device prevents the embarrassing wetness, odor and staining of natural urinary incontinence. One advantage of the disclosed male incontinence device is its unique elastic band fastening-system, which 1) prevents slippage and dislodgement (by keeping the disclosed male incontinence device secured below the level of the glans penis), 2) provides gentle urethral compression that decreases leakage without causing vascular compromise, 3) allows quick and easy application and removal, 4) is comfortable over a wide range of body motions, including strenuous exercise, 5) fits a wide range of phallic sizes, 6) self-adjusts to changing phallic dimensions, 7) is inconspicuous, unobtrusive and silent. Additionally, the disclosed male urinary incontinence device is compact, and may be carried in a wallet, pocket, glove compartment, etc. In addition, the disclosed male incontinence device is disposable.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A male urinary incontinence device comprising:
a generally square shaped pad, wherein the generally square shaped pad comprises:
a first corner located on the generally square shaped pad;
a second corner located on the generally square shaped pad;
a third corner located on the generally square shaped pad;
a forth corner located on the generally square shaped pad;
a first layer that is water absorbent;
a second layer attached to the first layer, and wherein the second layer is water impermeable;
a first guide line visibly located on the first layer, and extending from generally the first corner to generally the third corner;
a second guide line visibly located on the first layer, and extending from generally the second corner to generally the fourth corner;
an elastic band attached to the first layer at generally the third corner.

2. The male incontinence device of claim 1, further comprising:
a guide figure, visibly located on the first layer, situated between the first corner and the center, superimposed on the first guide line.

3. The male incontinence device of claim 1, wherein the first line is visibly distinguishable from the second line.

4. The male incontinence device of claim 3, wherein the first line is a solid line and the second line is a dashed line.

5. The male incontinence device of claim 2, wherein the guide figure is in the shape of a male phallus.

6. The male incontinence device of claim 1, wherein at least one plastic staple attaches the elastic band to the first layer.

7. The male incontinence device of claim 1, wherein an adhesive tape attaches the elastic band to the first layer.

8. The male incontinence device of claim 1, further comprising:
at least one oblong water permeable pocket containing a water absorbing material attached to the first layer near the first corner, and the length of the oblong pocket is generally parallel to the first line.

9. The male incontinence device of claim 8, further comprising:
a first oblong pocket containing a water absorbing material attached to the first layer near the first corner, the length of the first oblong pocket is generally parallel to the first line, and the first oblong pocket is located on a first side of the first line; and
a second oblong pocket containing a water absorbing material attached to the first layer near the first corner 26, the length of the second oblong pocket is generally parallel to the first line, and the second oblong pocket is located on a second side of the first line.

10. The male incontinence device of claim 8, wherein the water absorbing material is polyacrylamide powder.

11. A method of applying a male urinary incontinence device to a user's phallus, comprising:
orienting the male incontinence device such that a first layer is facing up, a second layer is facing down, and a solid line between the first and third corners is oriented perpendicular to an imaginary line connecting the user's hips, and an elastic band and third corner are oriented at generally the 12 o'clock position relative to the user;
moving the elastic band superiorly and off the first layer;
placing the phallus on the first layer generally along the first line and below the second line;
folding the male incontinence device generally at the second line and over the phallus;
folding a fourth corner over the phallus and adjacent to a third corner;
folding a second corner such that it is adjacent to the third corner;
looping the elastic band over the phallus and the male incontinence device such that the that elastic band is below glans;
creating a second loop by twisting the elastic band about 180 degrees; and
placing the second loop over and around the male incontinence device and phallus such that the second loop lies below the glans.

12. A method of packaging a male urinary incontinence device, comprising:
orienting the male incontinence device such that a first corner is pointed away from a packager;
folding the first corner over until it covers a third corner; flipping the device along the axis of the second line until the third corner points away from the user
rotating the device 180 degrees around its center until the third corner points toward the user
folding a second corner over until it covers the left half of the third corner, creating a fifth corner;
folding a fifth corner so that it generally meets a midpoint between a fourth corner and the third corner; and
folding a fourth corner toward the second corner, creating a compact triangular unit.
